(19) United States
(12) Patent Application Publication
(10) Pub. No.: US 2025/0128049 A1
(43) Pub. Date: Apr. 24, 2025

(54) CONTROLLER FOR A MEDICAL DEVICE

(71) Applicant: Abiomed, Inc., Danvers, MA (US)

(72) Inventors: Scott Corbett, Danvers, MA (US); Erik Ian Kroeker, Danvers, MA (US); Rebecca D'Agostine, Danvers, MA (US); Ramzi Hanania, Danvers, MA (US); Tyler Valentine, Danvers, MA (US)

(73) Assignee: Abiomed, Inc., Danvers, MA (US)

(21) Appl. No.: 18/922,668

(22) Filed: Oct. 22, 2024

Related U.S. Application Data
(60) Provisional application No. 63/592,521, filed on Oct. 23, 2023.

(51) Int. Cl. A61M 60/585; A61M 60/13; A61M 60/216; A61M 60/546; A61M 60/554

(52) U.S. Cl. CPC A61M 60/585; A61M 60/13; A61M 60/216; A61M 60/546; A61M 60/554; A61M 2205/18; A61M 2205/502

(57) ABSTRACT

Methods and apparatus for controlling a heart pump system are described. The heart pump system includes a first heart pump and a second heart pump and a controller. The controller includes a data interface configured to receive first data from the first heart pump and second data from the second heart pump, and at least one computer processor. The at least one computer process is programmed to display in a first portion of a user interface, an indication of the first data, display in a second portion of the user interface, an indication of the second data, and control an operation of the first heart pump and/or the second heart pump.

### RELATED APPLICATIONS

This application claims the benefit under 35 U.S.C. § 119(e) to U.S. Provisional Patent Applicant No. 63/592,521, filed Oct. 23, 2023, and titled “CONTROLLER FOR A MEDICAL DEVICE,” the entire contents of which is incorporated by reference herein.

### FIELD OF THE INVENTION

This disclosure relates to a controller for a medical device.

### BACKGROUND

Cardiovascular diseases are a leading cause of morbidity, mortality, and burden on global healthcare. A variety of treatment modalities have been developed for heart health, ranging from pharmaceuticals to mechanical devices and transplantation. Temporary cardiac support devices, such as heart pump systems (also referred to as “intracardiac blood pumps”), provide hemodynamic support and facilitate heart recovery. Intracardiac blood pumps have traditionally been used to temporarily assist the pumping function of a patient's heart during emergent cardiac procedures, such as a stent placement, performed after the patient suffers a heart attack, cardiac arrest, and/or cardiogenic shock. Intracardiac blood pumps also may be used to take the load off of a patient's heart to allow the heart to recover from such a cardiac procedure or from a heart attack, cardiac arrest, cardiogenic shock, or heart damage (e.g., caused by a viral infection). In that regard, an intracardiac blood pump can be introduced into the heart either surgically or percutaneously and used to deliver blood from one location in the heart or circulatory system to another location in the heart or circulatory system. For example, when deployed in the left heart, an intracardiac blood pump can pump blood from the left ventricle of the heart into the aorta. Likewise, when deployed in the right heart, an intracardiac blood pump can pump blood from the inferior vena cava into the pulmonary artery. Intracardiac pumps can be powered by a motor located outside of the patient's body via an elongate drive shaft (or drive cable) or by an onboard motor located inside the patient's body. Examples of such devices include the Impella® family of devices (Abiomed, Inc., Danvers, MA).

### SUMMARY

Described herein are systems and methods for a controller for a medical device support system, such as a cardiac support system. The controller may be configured to receive data from one or more medical devices (e.g., a right heart pump and a left heart pump) and control operation of the one or more medical devices.

In some embodiments, a controller for a heart pump system including a first heart pump and a second heart pump is provided. The controller includes a data interface configured to receive first data from the first heart pump and second data from the second heart pump, and at least one computer processor. The at least one computer processor is programmed to display in a first portion of a user interface, an indication of the first data, display in a second portion of the user interface, an indication of the second data, and control an operation of the first heart pump and/or the second heart pump.

In one aspect, the at least one computer processor is further programmed to display, in a third portion of the user interface, a first alert, the first alert determined based on the first data and/or the second data. In another aspect, the first alert is determined based on the first data and the at least one computer processor is further programmed to display, in a fourth portion of the user interface, a second alert, the second alert determined based on the second data. In another aspect, the first alert includes a recommendation to change an operation of first heart pump and/or the second heart pump.

In another aspect, the first data includes information describing operation of the first heart pump, and the second data includes information describing operation of the second heart pump. In another aspect, the information describing operation of the first heart pump includes one or more of a pump speed, a flow rate of blood through the pump, or a pressure measurement. In another aspect, the indication of the first data includes a first waveform and the indication of the second data includes a second waveform. In another aspect, the first waveform comprises a first pressure waveform and the second waveform comprises a second pressure waveform.

In another aspect, the controller further includes a communication interface configured to provide at least some of the first data and/or the second data to an auxiliary monitor for display. In another aspect, the at least one computer processor is further programmed to display on the user interface, at least one control element configured to receive user input, and control an operation of the first heart pump and/or the second heart pump in response to receiving user input via the at least one control element. In another aspect, controlling an operation of the first heart pump and/or the second heart pump comprises controlling a pump speed of the first heart pump and/or the second heart pump. In another aspect, controlling a pump speed of the first heart pump and/or the second heart pump is performed based, at least in part, on the first data and/or the second data. In another aspect, the first heart pump is configured to be placed in a left side of a heart of a patient, and the second heart pump is configured to be placed in the right side of the heart of the patient.

In some embodiments, a heart pump system is provided. The heart pump system includes a first heart pump configured to be placed in a left side of a heart of a patient, a second heart pump configured to be placed in a right side of the heart of the patient, and a multi-pump controller. The multi-pump controller is configured to control an operation of the first heart pump and/or the second heart pump, receive first data from the first heart pump and second data from the second heart pump, and display an indication of the first data and/or the second data on a user interface.

In one aspect, the heart pump system further includes an auxiliary monitor, and the multi-pump controller is further configured to provide at least some of the first data and/or the second data to the auxiliary monitor for display. In some embodiments, the heart pump system further includes a purge system configured to provide a purge fluid to the first heart pump and the second heart pump. In another aspect, the multi-pump controller is further configured to control an operation of the first heart pump and/or the second heart pump based, at least in part, on the first data and/or the second data. In another aspect, the multi-pump controller is configured to control an operation of the first heart pump and/or the second heart pump by changing a pump speed of the first heart pump and/or the second heart pump.

In some embodiments, a method of controlling a first heart pump and a second heart pump using a multi-pump controller is provided. The method includes receiving, from the first heart pump, first data associated with operation of the first heart pump, receiving, from the second heart pump, second data associated with operation of the second heart pump, displaying, on a first portion of a user interface, an indication of the first data, displaying, on a second portion of the user interface, an indication of the second data, and operating the multi-pump controller to control an operation of the first heart pump and/or the second heart pump.

In one aspect, operating the multi-pump controller to control an operation of the first heart pump and/or the second heart pump is based, at least in part, on the first data and/or the second data. In another aspect, operating the multi-pump controller to control an operation of the first heart pump and/or the second heart pump comprises changing a pump speed of the first heart pump and/or the second heart pump.

### BRIEF DESCRIPTION OF DRAWINGS

FIG. 1 shows an illustrative heart pump device that may be used to generate data for use with some embodiments.

FIG. 2 shows an illustrative heart pump system that includes a controller configured to control multiple heart pump devices, in accordance with some embodiments.

FIGS. 3A-3D illustrate portions of a user interface of a controller configured to display information from one or more connected heart pump devices, in accordance with some embodiments.

FIG. 4 is a flowchart of a process for displaying information from multiple heart pump devices on a user interface of a controller, in accordance with some embodiments.

### DETAILED DESCRIPTION

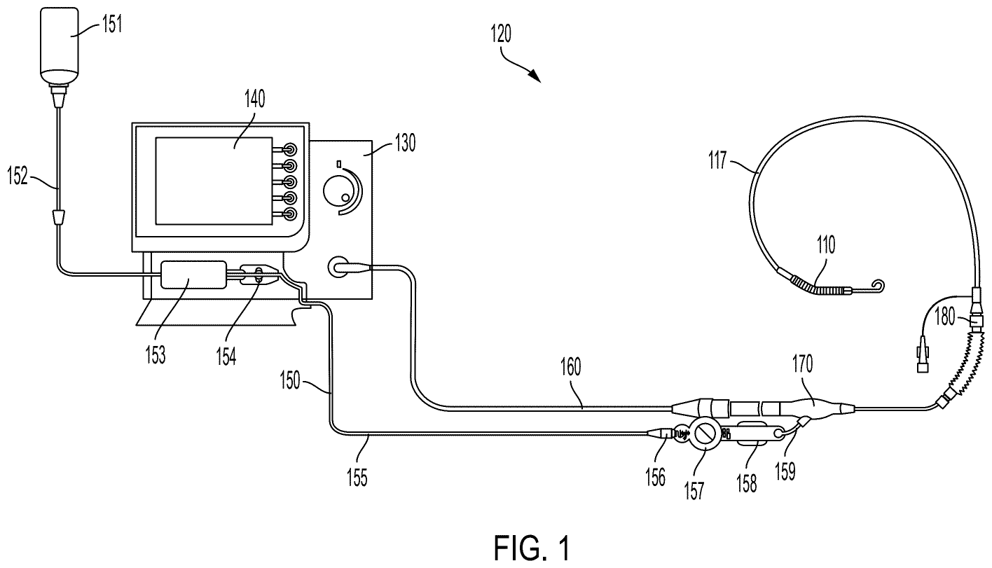

A circulatory support device (also referred to herein as a “heart pump” or simply a “pump”) may include a percutaneous, catheter-based device that provides hemodynamic support to the heart of a patient. As shown in FIG. 1, heart pump 110 may form part of a cardiac support system 100. Cardiac support system 100 also may include a controller 130 (e.g., an Automated Impella Controller®, referred to herein as an “AIC,” from ABIOMED, Inc., Danvers, Mass.), a display 140, a purge subsystem 150, a connector cable 160, a plug 170, and a repositioning unit 180. As shown, controller 130 may include display 140. Controller 130 may be configured to monitor and control operation of heart pump 110. During operation, purge subsystem 150 may be configured to deliver a purge fluid to heart pump 110 through catheter tube 117 to prevent blood from entering the motor (not shown) of the heart pump. In some implementations, the purge fluid is a dextrose solution (e.g., 5% dextrose in water with 25 or 50 IU/mL of heparin, although the solution need not include heparin in all embodiments). Connector cable 160 may provide an electrical connection between heart pump 110 and controller 130. Plug 170 may connect catheter tube 117, purge subsystem 150, and connector cable 160. In some implementations, plug 170 may include a storage device (e.g., a memory) configured to store, for example, operating parameters to facilitate transfer of the patient to another controller if needed. Repositioning unit 180 may be used to reposition heart pump 110 in the patient's heart.

As shown in FIG. 1, in some embodiments, the cardiac support system 100 may include a purge subsystem 150 having a container 151, a supply line 152, a purge cassette 153, a purge disc 154, purge tubing 155, a check valve 156, a pressure reservoir 157, an infusion filter 158, and a sidearm 159. Container 151 may, for example, be a bag or a bottle. As will be appreciated, in other embodiments the cardiac support system 100 may not include a purge subsystem. In some embodiments, a purge fluid may be stored in container 151. Supply line 152 may provide a fluidic connection between container 151 and purge cassette 153. Purge cassette 153 may control how the purge fluid in container 151 is delivered to heart pump 110. For example, purge cassette 153 may include one or more valves for controlling a pressure and/or flow rate of the purge fluid. Purge disc 154 may include one or more pressure and/or flow sensors for measuring a pressure and/or flow rate of the purge fluid. As shown, controller 130 may include purge cassette 153 and purge disc 154. Purge tubing 155 may provide a fluidic connection between purge disc 154 and check valve 156. Pressure reservoir 157 may provide additional filling volume during a purge fluid change. In some implementations, pressure reservoir 157 includes a flexible rubber diaphragm that provides the additional filling volume by means of an expansion chamber. Infusion filter 158 may help prevent bacterial contamination and air from entering catheter tube 117. Sidearm 159 may provide a fluidic connection between infusion filter 158 and plug 170. Although shown as having separate purge tubing and connector cable, it will be appreciated that in some embodiments, the cardiac support system 100 may include a single connector with both fluidic and electric lines connectable to the controller 130.

During operation, controller 130 may be configured to receive measurements from one or more pressure sensors (not shown) included as a portion of heart pump 110 and purge disc 154. Controller 130 may also be configured to control operation of the motor (not shown) of the heart pump 110 and purge cassette 153. As noted herein, controller 130 may be configured to control and measure a pressure and/or flow rate of a purge fluid via purge cassette 153 and purge disc 154. During operation, after exiting purge subsystem 150 through sidearm 159, the purge fluid may be channeled through purge lumens (not shown) within catheter tube 117 and plug 170. Sensor cables (not shown) within catheter tube 117, connector cable 160, and plug 170 may provide an electrical connection between components of the heart pump 110 (e.g., one or more pressure sensors) and controller 130. Motor cables (not shown) within catheter tube 117, connector cable 160, and plug 170 may provide an electrical connection between the motor of the heart pump 110 and controller 130. During operation, controller 130 may be configured to receive measurements from one or more pressure sensors of the heart pump 110 through the sensor cables (e.g., optical fibers) and to control the electrical power delivered to the motor of the heart pump 110 through the motor cables. By controlling the power delivered to the motor of the heart pump 110, controller 130 may be operable to control the speed of the motor,

Various modifications can be made to cardiac support system 100 and one or more of its components. For instance, one or more additional sensors may be added to heart pump 100. In another example, a signal generator may be added to heart pump 100 to generate a signal indicative of the rotational speed of the motor of the heart pump 110. As another example, one or more components of cardiac support system 100 may be separated. For instance, display 140 may be incorporated into another device in communication with controller 130 (e.g., wirelessly or through one or more electrical cables).

A heart pump (e.g., heart pump 110) may include a pressure sensor (e.g., an optical pressure sensor) configured to detect a pressure within the aorta of a patient's heart when the heart pump is properly positioned. The pressure signal sensed by the pressure sensor may be used, at least in part, to determine correct positioning of the heart pump within the patient's heart and/or to determine a blood flow rate through the heart pump when in operation. For instance, the pressure signal may be used in combination with a motor current signal received from a motor current sensor (not shown) and a set of stored values to determine a flow rate of blood through the heart pump. The differential pressure across the aortic valve may also indirectly be determined based on the pressure signal measuring the pressure in the aorta and the set of stored values.

Conventional medical devices include a controller and a display, which may be used to monitor and/or control one or more aspects of the operation of the medical device and/or the patient's physiology. In this regard, each device may be associated with a corresponding controller and display. The inventors have recognized and appreciated that when a patient is simultaneously using multiple medical devices (e.g., multiple heart pumps or a heart pump and an ECMO machine), each device is to be connected to its own controller, making a healthcare worker (e.g., a physician) monitor multiple controllers and displays to determine how the corresponding medical devices are operating. Some embodiments of the technology described herein relate to a controller and an associated display configured to receive data from multiple medical devices and display the data in a manner that provides a more intuitive and complete view of the operating parameters of multiple medical devices and/or a patient's physiological condition when using the medical devices. For example, a physician may be able to monitor operation of multiple heart pumps (e.g., a left side heart pump device and a right side heart pump device) on a single display rather than having to monitor multiple displays connected to individual medical devices. In some embodiments, the controller includes at least one processor configured to process the data received from the multiple medical devices. In some instances, the data from multiple medical devices may be combined in a way that is not possible when the data from each medical device is considered in isolation. For example, as described herein, the combined data may be analyzed via an algorithm that may provide alerts and/or a holistic recommendation based on the combined data.

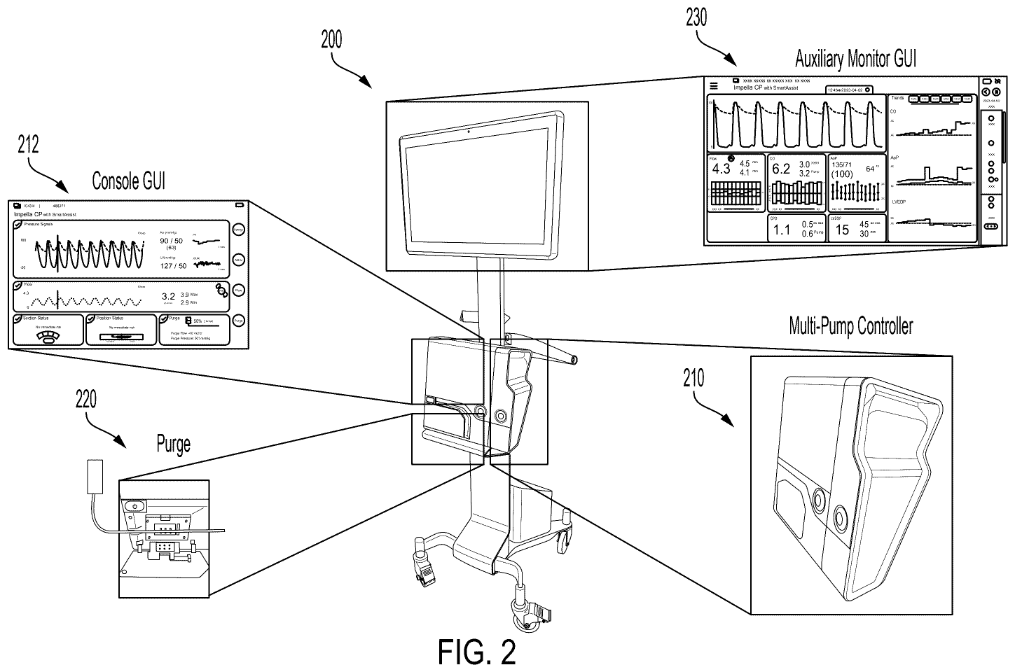

FIG. 2 illustrates a heart pump system 200 in accordance with some embodiments of the present disclosure. The heart pump system 200 includes a multi-pump controller 210, also referred to herein as a multi-pump controller 210 configured to control multiple heart pump devices (e.g. a right-side heart pump device and a left-side heart pump device) connected to the multi-pump controller 210. Multi-pump controller 210 may be configured to receive data (e.g., pump operation data, patient physiological data) from the connected heart pump devices and an indication of the received data may be displayed on a user interface 212 (e.g., a graphical user interface (GUI)) associated with the multi-pump controller 210. In some embodiments, user interface 212 may include one or more control elements that enable a user to control an operation of the heart pump devices connected to multi-pump controller 210. For instance, a healthcare provider may interact with one or more of the control element(s) to adjust a pump speed of one or more of the connected heart pump devices. In some embodiments, user interface 212 may be configured to display a recommendation for controlling one or more of the connected heart pump devices. For instance, a recommendation may be determined based, at least in part, on data received from the connected heart pump devices, and the recommendation may be displayed on the user interface 212. In response, a healthcare provider viewing the recommendation may determine whether to follow the recommendation and adjust the operation of the heart pump device(s) accordingly.

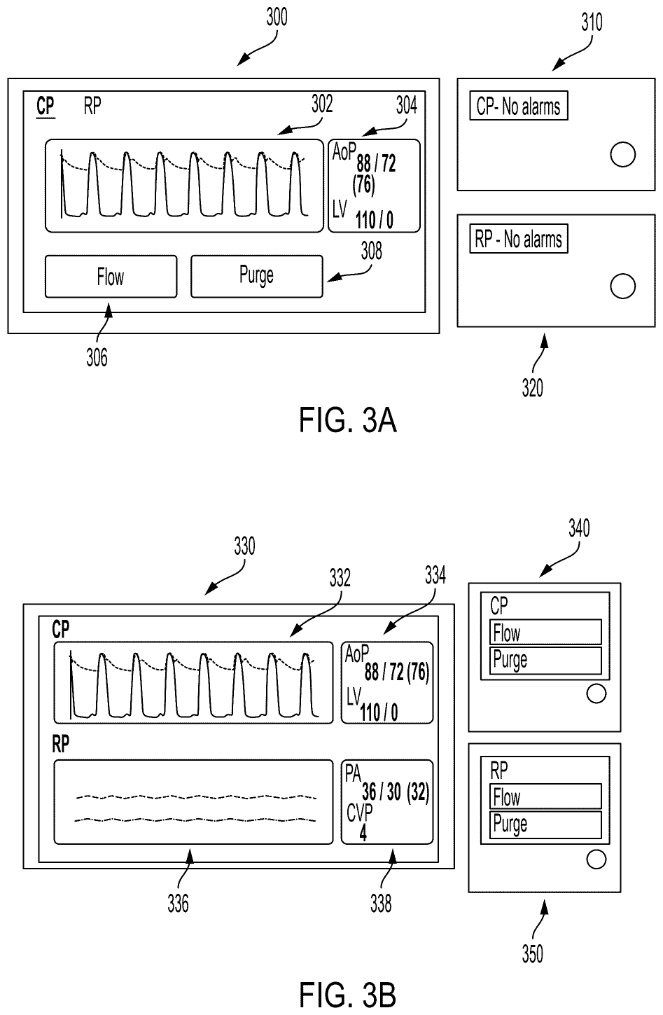

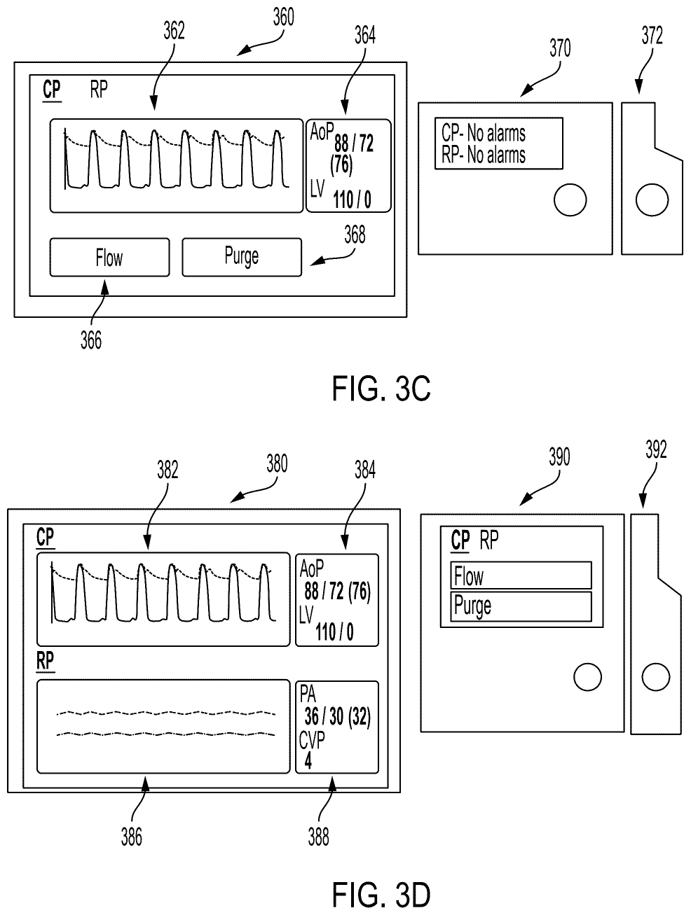

In the example of FIG. 2, a display on which user interface 212 is presented is integrated with multi-pump controller 210. It should be appreciated, however, that such a display may be provided separate from, but in communication with, multi-pump controller 210. As described in connection with FIGS. 3A-3D, user interface 212 may be configured to display data received from the connected heart pumps in a manner that facilitates a healthcare provider's monitoring of the patient within which the heart pumps are placed.

As shown in FIG. 2, heart pump system 200 may further include a purge system 220. Purge system 220 may be configured to provide a purge fluid to each of multiple connected heart pumps via, e.g., a lumen in a respective catheter coupled between the purge system 220 and the heart pump. In the example shown in FIG. 2, purge system 220 is integrated with multi-pump controller 210. However, it should be appreciated that in some embodiments, purge system 220 may be provided separately from multi-pump controller 210. As will be appreciated, in some embodiments, the controller may be connected to a single purge system that provides a purge fluid to each pump while in other embodiments, the controller may include first and second purge systems, each configured to provide a purge fluid to a respective pump.

As shown in FIG. 2, heart pump system 200 may further include an auxiliary monitor 230 communicatively coupled to multi-pump controller 210. Multi-pump controller 210 may be configured to provide data received from the connected heart pumps to auxiliary monitor 230 for further processing and/or display. For instance, auxiliary monitor 230 may include one or more computer processors that may be programmed to process the data received from the multi-pump controller 210. The data received from the multi-pump controller 210 and/or the processed data may be displayed by auxiliary monitor 230. In some embodiments, auxiliary monitor 230 may have a larger display compared to the display configured to show user interface 212. In some embodiments, auxiliary monitor 230 may have more processing power, storage resources, etc., compared to multi-pump controller 210, although in other embodiments, the auxiliary monitor 230 and the multi-pump controller 210 may be similarly configured in terms of processing power, storage resources, etc. In some embodiments, auxiliary monitor 230 may be communicatively coupled to one or more patient monitoring devices (e.g., a patient monitor, a respiratory monitor, a body temperature monitor, an extracorporeal membrane oxygenation (ECMO) machine, etc.) in addition to being communicatively coupled to multi-pump controller 210.

In some embodiments, multi-pump controller 210 and/or auxiliary monitor 230 may be configured to determine one or more hemodynamic signals, predict one or more hemodynamic signals, and/or evaluate whether an alert threshold has been met. In some embodiments, the auxiliary monitor 230 may display indications of data received from multi-pump controller 210 in a configurable user interface using one or more waveforms, values, trends and/or indicators. In still other embodiments, the auxiliary monitor may be configured to display patient data, such as data received from an electronic health record (EHR) stored in a hospital information system.

As described in more detail herein, a patient may have multiple cardiac support devices (e.g., a right side heart pump device and a left side heart pump device). Metrics that rely on information from both of the cardiac support devices may be determined and recommendations on how to modify operation of one or both of the cardiac support devices to optimize patient outcomes may be provided to guide treatment. For instance, a recommendation to increase or decrease support for one or both of the cardiac support devices may be determined, such as to maintain balanced support across both of the cardiac support devices. Algorithms for escalation or weaning may use one or more models that predict, based on current information from multiple information streams, how a change in the level of support may affect the cardiac and/or overall health of the patient (e.g., as the patient is working toward a particular goal, such as weaning off of a device). Such algorithms may be performed by one or more processors of multi-pump controller 210 and/or auxiliary monitor 230. In this way, heart pump system 200 may be configured to provide a physician or other healthcare provider a more integrated or complete view of a patient's health than can be obtained from viewing multiple displays associated with individual heart pump devices.

As will be appreciated, the multi-pump controller 210 may be configured in numerous different arrangements. For example, in some embodiments, a single unit may be configured to connect to multiple pumps (or other medical devices). In other embodiments, a single unit may be configured to connect to only a single medical device (e.g., a pump), with auxiliary units attachable thereto to connect additional medical devices.

FIGS. 3A-3D illustrate exemplary portions of a user interface (e.g., user interface 212) in accordance with some embodiments of the present disclosure. FIG. 3A illustrates a user interface 300 on which information based on data received from multiple connected heart pumps may be displayed. As shown, user interface 300 may include the ability to switch between data shown for a first heart pump (e.g., CP) and a second heart pump (e.g., RP). In the example shown in FIG. 3A, the data for heart pump CP is currently displayed on user interface 300. Example types of information that may be shown on user interface 300 include one or more waveforms 302 and numerical values, such as pressure values 304. User interface 300 may further include flow element 306 which, when selected by a user, may show information about blood flow through the currently displayed heart pump device (e.g., flow rate through heart pump CP in the example of FIG. 3A). User interface 300 may further include purge element 308 which, when selected by a user, may show information about the purge fluid being provided to the currently displayed heart pump device (e.g., from purge system 220). In some embodiments, user interface 300 may display information about one or more alarms associated with one or more of the heart pump devices (e.g., connected to multi-pump controller 210). In the example shown in FIG. 3A, user interface 300 includes a first alarm status portion 310 corresponding to a first heart pump device (e.g., connected to multi-pump controller 210) and a second alarm status portion 320 corresponding to a second heart pump device connected to multi-pump controller 210. Providing alarm information about each of the multiple heart pumps connected to a controller (e.g., multi-pump controller 210) on the same user interface 300 may facilitate monitoring of a patient's treatment by a healthcare provider.

FIG. 3A also illustrates an example in which a single unit includes multiple connections with a single small console screen.

FIG. 3B illustrates a user interface 330 on which information based on data received from multiple connected heart pumps may be displayed, in this instance with a single, large console screen and a multi-pump device having a single console with multiple connections. In the example shown in FIG. 3B waveforms and numerical values for each of the multiple connected heart pump devices are shown on user interface 330 at the same time. For instance a first waveform 332 and numerical values 334 associated with heart pump device CP are shown on a top portion of user interface 330, while a second waveform 336 and numerical values 338 associated with heart pump device RP are shown in a bottom portion of user interface 330. Due, for example, to the limited real estate available on user interface 330, blood flow data and purge system data for each of the connected heart pump devices may be displayed in separate portions 340 and 350 of user interface 330 as shown in FIG. 3B. It should be appreciated that the user interfaces described herein in connection with some embodiments of the present disclosure may be configurable such that a particular healthcare provider may provide a view of data from the multiple connected heart pump devices as desired.

FIG. 3C illustrates a user interface 360, which includes information similar to that shown in user interface 300 of FIG. 3A. For instance, user interface 360 includes the ability to switch between data shown for a first heart pump (e.g., CP) and a second heart pump (e.g., RP). In the example shown in FIG. 3C, the data for heart pump CP is currently displayed on user interface 360. Example types of information that may be shown on user interface 360 include one or more waveforms 362 and numerical values, such as pressure values 364. User interface 360 may further include flow element 366 which, when selected by a user, may show information about blood flow through the currently displayed heart pump device (e.g., flow rate through heart pump CP in the example of FIG. 3C). User interface 360 may further include purge element 368 which, when selected by a user, may show information about the purge fluid being provided to the currently displayed heart pump device (e.g., from purge system 220). In some embodiments, user interface 360 may display information about one or more alarms associated with one or more connected heart pump devices. In the example shown in FIG. 3C, user interface 360 includes an indication of both a first alarm status for a first heart pump device (e.g., heart pump device CP) and a second alarm status for a second heart pump device (e.g., heart pump device RP) in a single portion (e.g., for a single/main controller 370) of the user interface. Providing alarm information about each of the multiple heart pumps (e.g., connected to multi-pump controller 210) on the same user interface 360 may facilitate monitoring of a patient's treatment by a healthcare provider. User interface 360 may further include an additional selectable element (e.g., for additional auxiliary modules 372), which may be used to display additional information when selected by a user.

As will be appreciated in view of FIG. 3C, the multi-pump controller may include a main controller 370 to which additional auxiliary modules 372 may be connected to connect additional pumps.

FIG. 3D illustrates a user interface 380, which includes information similar to that shown in user interface 330 of FIG. 3B. FIG. 3D illustrates a user interface 380 in which information based on data received from multiple connected heart pumps may be displayed. In the example shown in FIG. 3D, waveforms and numerical values for each of the multiple heart pump devices are shown on user interface 380 at the same time. For instance a first waveform 382 and numerical values 384 associated with heart pump device CP are shown on a top portion of user interface 380, while a second waveform 386 and numerical values 388 associated with heart pump device RP are shown in a bottom portion of user interface 380. Due, for example, to the limited real estate available on user interface 380, blood flow data and purge system data for each of the connected heart pump devices may be displayed in a separate portion (e.g., for a single/main controller 390) of user interface 380 as shown in FIG. 3D. User interface 380 may further include an additional selectable element (e.g., for an auxiliary device 392), which may be used to display additional information when selected by a user. In FIG. 3D, similar to FIG. 3C, the multi-pump controller may include a main controller 390 and an auxiliary device 392 which can be connected to the main controller.

Although shown with auxiliary devices to connect additional pumps to the main controller (to form the multi-pump controller), it will be appreciated that the multi-pump controller may include auxiliary modules to allow one or more other medical devices (e.g., an echo device, an EMCO device, an ultrasound device) to be connected to the main device. As will be appreciated in view of the above, in such embodiments, the multi-device controller may be configured to process information from the multiple medical devices to provider alerts and/or a holistic recommendation based on the information received from the devices and/or from patient data coming from an electronic health record (HER). In this regard, the multi-device controller may be equipped with modularity to allow a clinician to create a controller to fit a particular patient's needs and be able to receive and process the corresponding data.

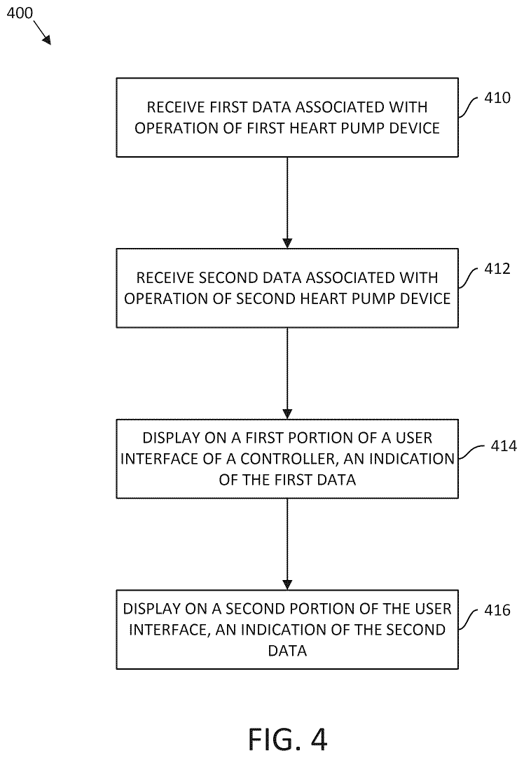

FIG. 4 is a flowchart of a process 400 for displaying, on a user interface (e.g., user interface 212), data from multiple connected heart pump devices, in accordance with some embodiments. In act 410, first data associated with operation of a first heart pump device may be received (e.g., by multi-pump controller 210). The first data received from the first patient monitoring device may include physiological data (e.g., pulse rate) for a patient with which the first heart pump device is associated and/or device data (e.g., pump speed) associated with operation of the first heart pump device. Additionally, the first data may be measured directly by the first heart pump device or may be derived based on data measured by the first heart pump device.

Process 400 may then proceed to act 412, where second data associated with operation of a second heart pump device may be received (e.g., by multi-pump controller 210). The second data received from the second heart pump device may include physiological data (e.g., pulse rate) for a patient with which the second heart pump device is associated and/or device data (e.g., pump speed) associated with operation of the second heart pump device. Additionally, the second data may be measured directly by the second heart pump device or may be derived based on data measured by the second heart pump device.

Process 400 may then proceed to act 414, where an indication of the first data may be displayed on a first portion of a user interface. The indication of the first data may take any suitable form including, but not limited to, one or more graphs, charts, numerical values, waveforms, trends and the like. In some embodiments, the indication of the first data may be a representation of the first data as received from the first heart pump device. In some embodiments, the indication of the first data may be a processed version of the first data received from the first heart pump device. For instance, the first data may be provided as input to a trained machine learning model, and the output of the trained machine learning model may be displayed on the user interface as the indication of the first data.

Process 400 may then proceed to act 416, where an indication of the second data may be displayed on a second portion of a user interface. The indication of the second data may take any suitable form including, but not limited to, one or more graphs, charts, numerical values, waveforms, trends and the like. In some embodiments, the indication of the second data may be a representation of the second data as received from the second heart pump device. In some embodiments, the indication of the second data may be a processed version of the second data received from the second heart pump device. For instance, the second data may be provided as input to a trained machine learning model, and the output of the trained machine learning model may be displayed on the user interface as the indication of the second data. The indication of the first data and the indication of the second data may be displayed in the same form or a different form on the user interface. For instance, the indication of the first data may be displayed as one or more graphs in the first portion of the user interface and the indication of the second data may be displayed as one or more numerical values in the second portion of the user interface.

Having thus described several aspects and embodiments of the technology set forth in the disclosure, it is to be appreciated that various alterations, modification, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above, and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A controller for a heart pump system, the heart pump system including a first heart pump and a second heart pump, the controller comprising:
a data interface configured to receive first data from the first heart pump and second data from the second heart pump; and
at least one computer processor programmed to:
display in a first portion of a user interface, an indication of the first data;
display in a second portion of the user interface, an indication of the second data; and
control an operation of the first heart pump and/or the second heart pump.

2. The controller of claim 1, wherein the at least one computer processor is further programmed to:
display, in a third portion of the user interface, a first alert, the first alert determined based on the first data and/or the second data.

3. The controller of claim 2, wherein the first alert is determined based on the first data and wherein at least one computer processor is further programmed to:
display, in a fourth portion of the user interface, a second alert, the second alert determined based on the second data.

4. The controller of claim 2, wherein the first alert includes a recommendation to change an operation of first heart pump and/or the second heart pump.

5. The controller of claim 1, wherein
the first data includes information describing operation of the first heart pump, and
the second data includes information describing operation of the second heart pump.

6. The controller of claim 5, wherein the information describing operation of the first heart pump includes one or more of a pump speed, a flow rate of blood through the first heart pump, or a pressure measurement.

7. The controller of claim 1, wherein the indication of the first data includes a first waveform and the indication of the second data includes a second waveform.

8. The controller of claim 7, wherein the first waveform comprises a first pressure waveform and the second waveform comprises a second pressure waveform.

9. The controller of claim 1, further comprising:
a communication interface configured to provide at least some of the first data and/or the second data to an auxiliary monitor for display.

10. The controller of claim 1, wherein the at least one computer processor is further programmed to:
display on the user interface, at least one control element configured to receive user input, and
control an operation of the first heart pump and/or the second heart pump in response to receiving user input via the at least one control element.

11. The controller of claim 1, wherein controlling an operation of the first heart pump and/or the second heart pump comprises controlling a pump speed of the first heart pump and/or the second heart pump.

12. The controller of claim 1, wherein controlling a pump speed of the first heart pump and/or the second heart pump is performed based, at least in part, on the first data and/or the second data.

13. The controller of claim 1, wherein
the first heart pump is configured to be placed in a left side of a heart of a patient, and
the second heart pump is configured to be placed in a right side of the heart of the patient.

14. A heart pump system, comprising:
a first heart pump configured to be placed in a left side of a heart of a patient;
a second heart pump configured to be placed in a right side of the heart of the patient; and
a multi-pump controller configured to:
control an operation of the first heart pump and/or the second heart pump;
receive first data from the first heart pump and second data from the second heart pump; and
display an indication of the first data and/or the second data on a user interface.

15. The heart pump system of claim 14, further comprising:
an auxiliary monitor,
wherein the multi-pump controller is further configured to provide at least some of the first data and/or the second data to the auxiliary monitor for display.

16. The heart pump system of claim 14, further comprising:
a purge system configured to provide a purge fluid to the first heart pump and the second heart pump.

17. The heart pump system of claim 14, wherein the multi-pump controller is further configured to control an operation of the first heart pump and/or the second heart pump based, at least in part, on the first data and/or the second data.

18. The heart pump system of claim 14, wherein the multi-pump controller is configured to control an operation of the first heart pump and/or the second heart pump by changing a pump speed of the first heart pump and/or the second heart pump.

19. A method of controlling a first heart pump and a second heart pump using a multi-pump controller, the method comprising:
receiving, from the first heart pump, first data associated with operation of the first heart pump;
receiving, from the second heart pump, second data associated with operation of the second heart pump;
displaying, on a first portion of a user interface, an indication of the first data;
displaying, on a second portion of the user interface, an indication of the second data; and
operating the multi-pump controller to control an operation of the first heart pump and/or the second heart pump.

20. The method of claim 19 wherein operating the multi-pump controller to control an operation of the first heart pump and/or the second heart pump is based, at least in part, on the first data and/or the second data.

21. The method of claim 19, wherein operating the multi-pump controller to control an operation of the first heart pump and/or the second heart pump comprises changing a pump speed of the first heart pump and/or the second heart pump.
